(19) United States
(12) Patent Application Publication
(10) Pub. No.: US 2025/0154195 A1
(43) Pub. Date: May 15, 2025

(54) NOVEL BETULIN DERIVATIVES, AND THEIR MANUFACTURING METHOD AND USE AS SURFACTANT

(71) Applicant: Sang Hyun PYO, Daejeon (KR)

(72) Inventors: Sang Hyun PYO, Daejeon (KR); Yoon Sun PYO, Uppsala (SE)

(21) Appl. No.: 18/833,370

(22) PCT Filed: Feb. 2, 2023
(86) PCT No.: PCT/KR2023/001507

§ 371 Date: Jul. 24, 2024

Related U.S. Application Data
(60) Provisional application No. 63/390,311, filed on Jul. 19, 2022.
(60) Provisional application No. 63/305,737, filed on Feb. 2, 2022.

(51) Int. Cl. C07J 63/00; C11D 1/66

(52) U.S. Cl. CPC C07J 63/008; C11D 1/66

(57) ABSTRACT

Betulin is a natural pentacyclic triterpene, and is contained in birch tree, especially its outer bark. Betulin can be used as a hydrophobic building block for production of biobased surfactants. This invention is related to amphiphilic betulin derivatives, and their efficient production method. And resulting materials can be used as biobased surfactants.

### TECHNICAL FIELD

This invention relates to hydrophilic moieties-derivatized betulin derivatives, and their production method from betulin on the one or two hydroxyl groups by chemical- or bio-catalysis. The invention further related to the production method of these betulin derivatives from betulin functionalized or protected on the primary or secondary hydroxyl group by oxidation, etherification, formate formation, and carbonation. The resulting materials consisted of a hydrophobic betulin and one or two hydrophilic moieties, can be used as surfactant with amphiphilic properties.

### BACKGROUND ART

Concerning pollution of the environment and everyday exposure to hazardous chemicals, it may be possible to avoid or greatly reduce exposure of people and the environment to hazardous chemicals by introducing new design concepts into the de-velopment of new products. Some chemicals can cause serious damage to human health and the environment, and are classified certain substances according to their substance groups such as alkylphenol and their derivatives, phthalates, and iso-cyanates. Among these, alkylphenols (APs), and in particular their polyethoxylated derivatives (alkylphenol ethoxylates, APEOs), deserve particular attention as commercial surfactants. By far the most commercially important AP is nonylphenol (NP) which is used primarily to produce nonylphenol ethoxylates (NPEOs) surfactants for a wide variety of applications and consumer products: paints and latex paints, adhesives, inks, washing agents, formulation of pesticides (emulsions), paper industry, textile and leather industry, petroleum recovery chemicals, metal working fluids, personal care products, cleaners and detergents, etc.

Despite the fact that metabolites of APEOs are classified as hazardous substances, they continue to be released into the environment from a variety of sources and are not usually monitored. Furthermore, NP has been known as an endocrine disruptor in humans and animals. Because of similarity of chemical structures (Scheme 1), NPs can act like the female hormone 17β-estradiol by binding to the estrogen receptor and displacing 17β-estradiol in a competitive manner.

Here, the new design and sustainable concept are required to replace the hazardous APEO by a greener and safer alternative having similar functionality. As an alternative to NP and 17β-estradiol, a triterpene, natural betulin can be used as the hydrophobic head backbone for introducing amphipathic compounds with hydrophilic side chain such as polyols, polyolamines, and saccharides.

Surfactants (surface active agents, emulsifiers also called detergents) reduce the surface tension between two immiscible phases, or one phase in another phase, and are used in products and applications where increased solubility is required. There are amphiphilic consisted of a hydrophilic portion which is soluble in water (“head”) and a hydrophobic portion that is soluble in oil (“tail”). In aqueous solution, the soluble surfactant will therefore spontancously form aggregates (micelles), where the hydrophobic part is directed inward away from the aqueous phase while the hydrophilic part is directed outward in the direction of the aqueous phase. This process is called solubilization, and the lowest surfactant concentration at which micelles are formed is called the critical micelle concentration (CMC). CMC is an important property of surfactants. Above the CMC, any additional surfactant added to the system becomes micelles. Prior to reaching CMC, surface tension varies strongly with surfactant concentration. After reaching the CMC, the surface tension remains relatively constant or changes with a lower slope. The CMC is directly dependent on surfactant structure, of which increasing the alkyl chain length reduces CMC and favors surfactant adsorption. This dependence has direct and substantial consequences for surfactant selection and design. Therefore, longer alkyl chains promote adsorption and aggregation, so lower concentrations of surfactant are required to achieve a given effect. Consequently, the combination of long alkyl chains with long heads is often advantageous with respect to surfactant functionality. As some examples of CMC for non-ionic surfactants, Triton X-100 (NPEO, 0.2 mM), Brij 35 (0.1 mM), Tween 20 (0.08 mM), sucrose monolaurate (0.45 mM), and fructose oleate (0.4 mM) were found in literatures.

Betulin is a pentacyclic triterpene of lupane type: lup-20 (29)-en-36,28-diol (CAS no. 473-98-3) (Scheme 1), and can be found in large amount (up to 20-35% of dry outer bark weight) depending on the tree species of birch. Silver birch (Betula pendula) is widely distributed in the northern hemisphere, and is of great commercial significance as it constitutes the dominant hardwood tree species used for pulp production. It is leading to the production of considerable amounts of birch bark as a residual by-product from log debarking, usually burned for energy production. Debarking, however, leads to wood loss and yield reduction. De-resination of birch wood in Kraft pulping is especially difficult because birch contains high amounts of unsaponifiable components, betulin being a major unsaponifiable component.

Betulin and betulinic acid have been the subject of intensive research due to their high pharmacological properties such as antiseptic, antiviral, anti-inflammatory, hep-atoprotective, and anticancer activity. Furthermore the research has been extended into the production of polyesters and polyurethanes from betulin.

Nevertheless, its utilization on the bio-surfactant production hasn't been employed, and can be expected as biodegradable and biocompatible chemicals with great po-tentials such as biobased economy, alternative to existing hazard materials, and use of abundant by-product from forest industry.

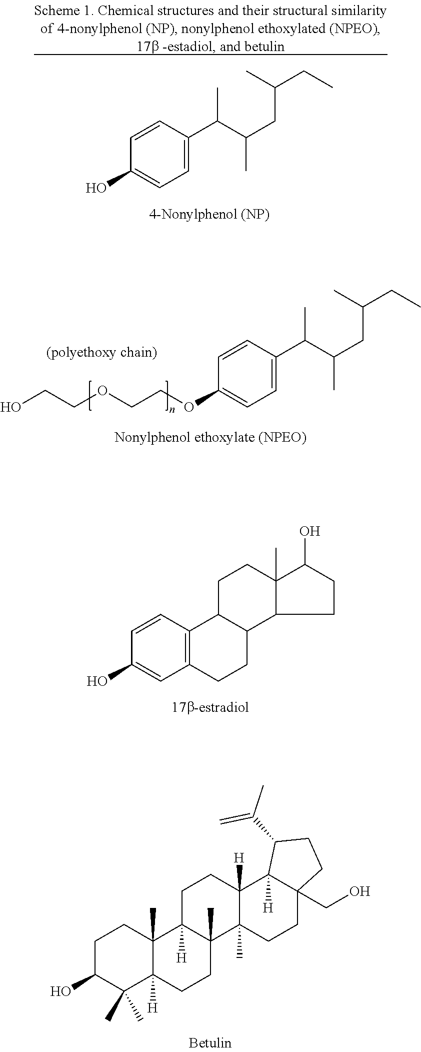

Therefore, based on the high hydrophobic structural feature, betulin can be a promising candidate of hydrophobic head for producing new bio-surfactants, and for replacing petroleum-based surfactants, which are generally toxic and difficult to break down through the action of microorganisms.

In prior arts, there have been many reports related to derivatization and/or oxidation of betulin to improve its biological activities. Many mono-, di-, and tri-terpenoid compounds are glycosides with the sugars linked to the active groups. Various betulin glycosides are isolated from plants and synthesized by chemical and biotechnology process. These compounds were evaluated on the bioactivity and cytotoxicity, however, the direct use of these compounds as surfactant wasn't found in literature.

In general, di-glycosylated betulin is prepared by glycosylation of two hydroxyl groups of betulin, however, to obtain mono-glycosylated betulin or mono-glycosylated betulin derivatives, the selective protection or derivatization at the primary alcohol of betulin is required. And, betulin has been polyethoxylated at both hydroxyl groups in only limited examples by Helmut Schlaad, while mono-(poly) ethoxylation of betulin derivatives wasn't found in literature.

Also, betulin derivatives functionalized with carbonate, chloroformate or acyl chloride groups can be used to selectively produce amphiphilic betulin moieties derivatized with polyethylenglycol (PEG), polyetheramine (PEA), polypropyleneglycol (PPG), glycosyl, saccharide, glucosamine, chitosan, etc.

In general, to obtain mono-hydrophilic betulin derivatives, the selective protection or derivatization at the primary alcohol is required.

### DISCLOSURE OF INVENTION

### Technical Problem

The present invention provides a novel compound that can be used as a naturally-derived environmentally friendly surfactant.

The present invention provides a method for synthesizing a novel compound from betulin.

The present invention provides a surfactant comprising the novel compound.

The present invention provides various functional materials, mono- and di-hydrophilic moiety derivatized betulin derivatives.

Further, this provides a facile process for manufacturing mono- and di-hydrophilic moiety derivatized betulin from betulin.

Further, this provides a facile process for manufacturing mono-hydrophilic moiety derivatized betulin derivatives from betulin through protected or derivatized betulin derivatives.

Further, this provides a facile process for manufacturing mono-hydrophilic moiety derivatized betulin derivatives from betulin through carbonated betulin derivatives.

Further, the protected or derivatized betulin derivatives can be obtained from catalytic oxidation, esterification, etherification or carbonation of primary hydroxyl group in betulin, but not limited. The protected or derivatized betulin derivatives are not limited on the oxidized or carbonated betulin, and include esterified and etherified betulin with acyl or alkyl derivatives.

Further, mono-hydrophilic moiety at C3-hydroxy group of betulin can be obtained from the protected or derivatized betulin derivatives.

Further, mono-hydrophilic moiety at C28-hydroxy group of betulin can be obtained from the protected or derivatized betulin derivatives.

Further, di-hydrophilic moiety at C28- and C3-hydroxy group of betulin can be obtained from the protected or derivatized betulin derivatives.

Further, mono-hydrophilic moiety derivatization of protected or derivatized betulin derivatives can be performed by chemical or bio-catalysis.

Further, di-hydrophilic moiety derivatization of betulin can be performed by chemical or bio-catalysis.

Further, hydrophilic moieties can be polyethylenglycol (PEG), polyetheramine (PEA), polypropyleneglycol (PPG), glycosyl, saccharide, glucosamine, chitosan, etc.

Further, mono-hydrophilic moiety derivatized betulin derivatives can be used as a surfactant with amphiphilic structure and properties.

Further, di-hydrophilic moiety derivatized betulin derivatives can be used as a surfactant with amphiphilic structure and properties.

### Solution to Problem

Therefore, an aspect of the present invention is to provide a compound having formula (1):

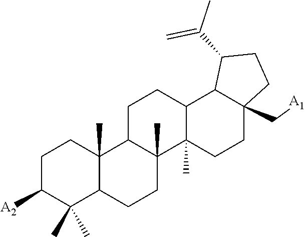

Wherein,
A₁ and A₂ is independently W₁;
W₁ is

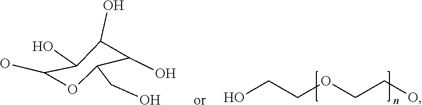

n is an integer of 1 to 100,
when one of A₁ or A₂ is W₁ and the other one is independently selected from H, (═O) OH, —OCOR₁, —OCOOR₂, —OR₃, and —OH,
R₁ and R₂ is H; or
R₁, R₂ and R₃ is independently selected from C₁-C₂₀ alkyl, allyl groups and halide group.

The compound of formula (1) is a betulin derivative of formula (1-1), (1-2), (1-3), or (1-4):

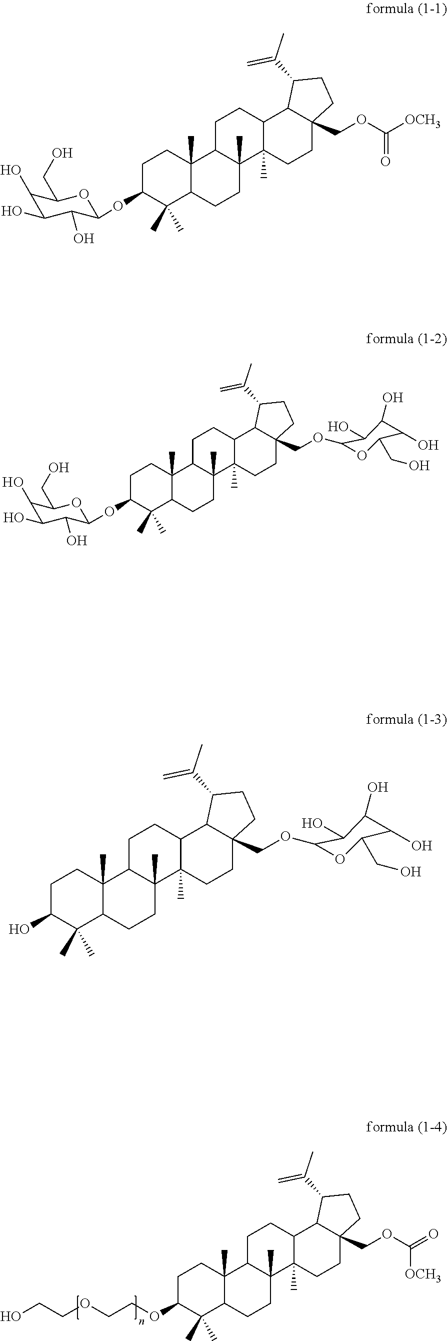

An aspect of the present invention is to provide a compound having formula (2):

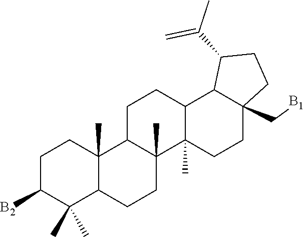

Wherein,
B₁ or B₂ is W₂;
W₂ is

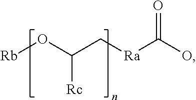

Ra is amine (NH) or oxygen (O),
Rb is amine (NH₂) or hydroxyl (—OH),
Rc is H, or C1-C3 alkyl,
n is an integer of 1 to 100,
when one of B₁ or B₂ is W₂, the other one is independently selected from H, (═O) OH, —OCOR₁, —OCOOR₂, —OR₃, and —OH,
R₁, R₂ and R₃ is independently is selected from C₁-C₂₀ alkyl, allyl groups and halide group.

The compound of formula (2) is a betulin derivative of formula (2-1), (2-2), (2-3), or (2-4):

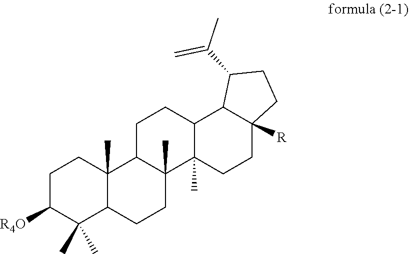

Wherein, R₄ is independently selected from —COOH, —COR₅, —COOR₆, and —COR₇COR₈,
R₅, R₆ and R₈ is independently hydrophilic moieties, R, is alkyl.

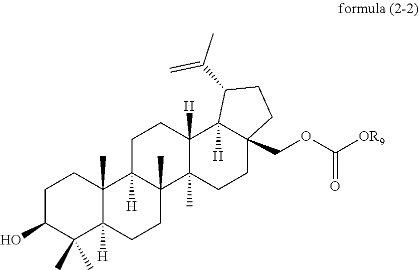

Wherein, R₉ is hydrophilic moieties, the hydrophilic moieties is selected from polyether glycol (PEG), polyetheramine (PEA), sugar, saccharide, oligo-saccharide, glucosamine, chitosan, and oligo-chitosan.

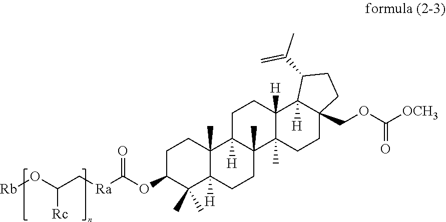

Wherein,
Ra is O,
Rb is OH or H,
Rc is H or CH₃ for hydrophilic moieties,
Wherein, Rc is H for PEG; or
Rc is CH₃ for PPG; or
Rc is H or CH₃ for mixture of PEG and PPG; or
Ra is NH, Rb is NH₂, Rc is alkyl for PEA

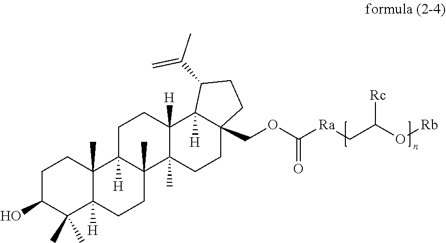

Wherein,
Ra is O,
Rb is OH or H,
Rc is H or CH₃ for hydrophilic moieties,
Wherein, Rc is H for PEG; or
Rc is CH₃ for PPG; or
Rc is H or CH₃ for mixture of PEG and PPG; or
Ra is NH, Rb is NH₂, Rc is alkyl for PEA.

An aspect of the present invention is to provide a method for manufacturing a compound, the method comprising: preparing a betulin; and glycosylation or alkoxylation of the betulin.

The method further comprising preparing an intermediate from the betulin; and glycosylation or alkoxylation of the intermediate.

The preparing the intermediate comprises derivatizing the betulin with acid, ester, carbonate or ether group at a primary hydroxyl group of betulin by oxidation, esterification, carbonation or etherification.

The glycosylation is performed with glycosyl donors by chemical or bio-catalysis.

The alkoxylation is performed with alkoxylation agents,

The alkoxylation agents is selected from among ethylene oxide, propylene oxide, and their mixture.

An aspect of the present invention is to provide a method for manufacturing a compound, the method comprising: preparing a betulin; preparing a first intermediate from the betulin; and functionalizing the first intermediate with hydrophilic moieties.

The method further comprising preparing a second intermediate from first intermediate; and functionalizing the second intermediate with hydrophilic moieties, wherein the preparing the second intermediate comprises activating C3-hydroxyl group of the first intermediate to chloroformate.

C3-hydroxyl group of the betulin is functionalized with a hydrophilic group in the step of functionalizing the second intermediate.

C28-hydroxyl group of the betulin is functionalized with a hydrophilic group in the step of functionalizing the first intermediate.

The hydrophilic moieties is selected from polyether glycol (PEG), polyetheramine (PEA), polypropyleneglycol (PPG), sugar, saccharide, oligo-saccharide, glucosamine, chitosan, and oligo-chitosan.

An aspect of the present invention is to provide a surfactant composition comprising the compound.

### Advantageous Effects of Invention

The present invention provides mono- and di-hydrophilic moiety derivatized betulin derivatives which can be used as a surfactant with amphiphilic structure and properties.

### BRIEF DESCRIPTION OF DRAWINGS

FIG. 1 is FT-IR spectrum of (A) Betulin, (B) Betulin carbonate, (C) Betulin-carbonate-formate, and (D) Betulin-carbonate-polyether (PEA).

FIG. 2 is FT-IR spectrum of (A) Betulin, (B) Betulin carbonate, (C) Betulin-carbonate-polyether (PEG).

### BEST MODE FOR CARRYING OUT THE INVENTION

A more complete appreciation of the invention, and many of the attendant ad-vantages thereof, will be readily apparent as the same become better understood by reference to the following detailed description.

Accordingly, the present invention relates to various functional materials, mono-hydrophilic moiety derivatized betulin derivatives prepared.

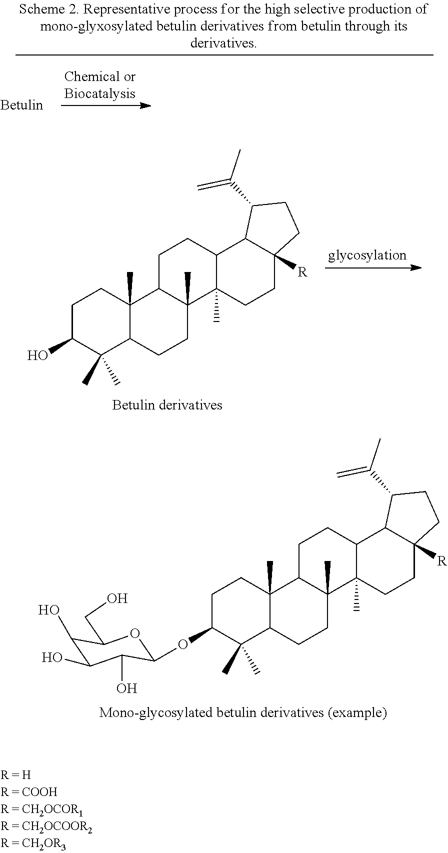

The invention relates to a process for the high selective production of mono-glycosylated betulin derivatives from betulin through its derivatives (Scheme 2).

Betulin can be derivatized with acid, ester, carbonate or ether group at the primary hydroxyl group by oxidation, esterification, carbonation and etherification (Scheme 2). The resulting derivatives can react with glycosyl donors by chemical or bio-catalysis (Scheme 2). The resulting mono-glycosylated betulin derivatives can be used as a surfactant with amphiphilic structure and properties.

Furthermore, the mono- and di-glycosylation of betulin can be performed with glycosyl donors by chemical or bio-catalysis (Scheme 3). The resulting di-glycosylated betulin can be used as a surfactant.

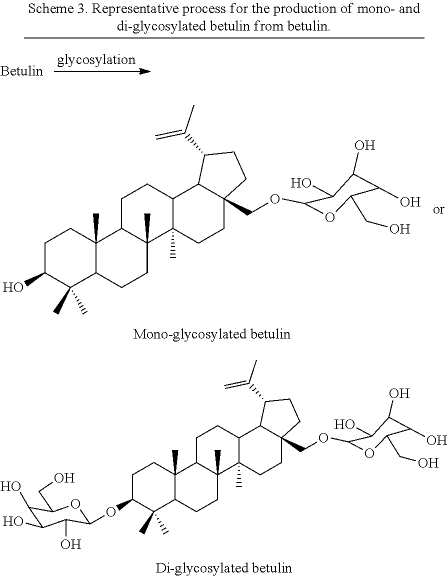

Accordingly, the present invention relates to various functional materials, mono-(poly) alkoxylated betulin derivatives prepared.

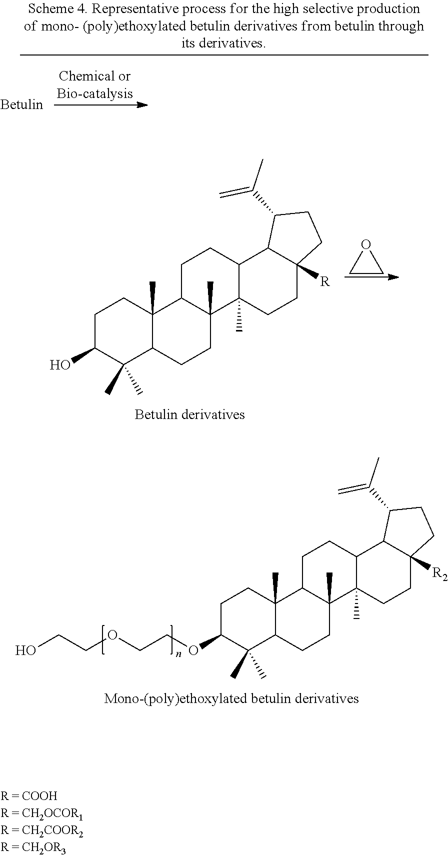

The invention relates to a process for the high selective production of mono-(poly) alkoxylated betulin derivatives from betulin through its derivatives (Scheme 4).

Betulin can be derivatized with acid, ester, carbonate or ether group at the primary hydroxyl group by oxidation, esterification, carbonation and etherification (Scheme 4). The resulting derivatives can react with alkoxylation agents such as ethylene oxide, propylene oxide, and their mixture in presence of base catalyst at a temperature from −30° C. to 200° C. for the high selective production of mono-(poly)alkoxylated betulin derivatives (Scheme 4).

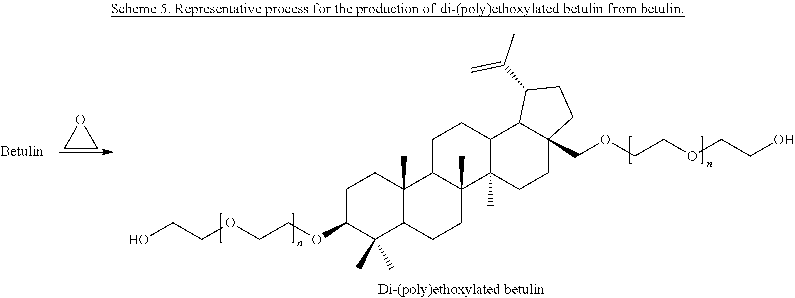

Furthermore, di-(poly)alkoxylated betulin can be used as a surfactant. The di-(poly)alkoxylation of betulin can be performed by using alkoxylation agents such as ethylene oxide, propylene oxide, and their mixture in present a catalyst at a temperature from −30° C. to 200° C. (Scheme 5).

The invention relates to a process for the high selective production of mono-hydrophilic betulin derivatives at C3-hydroxy group from betulin through its derivatives (Scheme 6). Betulin can be derivatized with acid, ester, carbonate or ether group at the primary hydroxyl group at C28 by oxidation, esterification, carbonation and etherification (Scheme 6). The resulting derivatives can be further functionalized at C3-hydroxy group with hydrophilic moieties by chemical or bio-catalysis (Scheme 6). The resulting mono-hydrophilic betulin derivatives can be used as a surfactant with amphiphilic structure and properties.

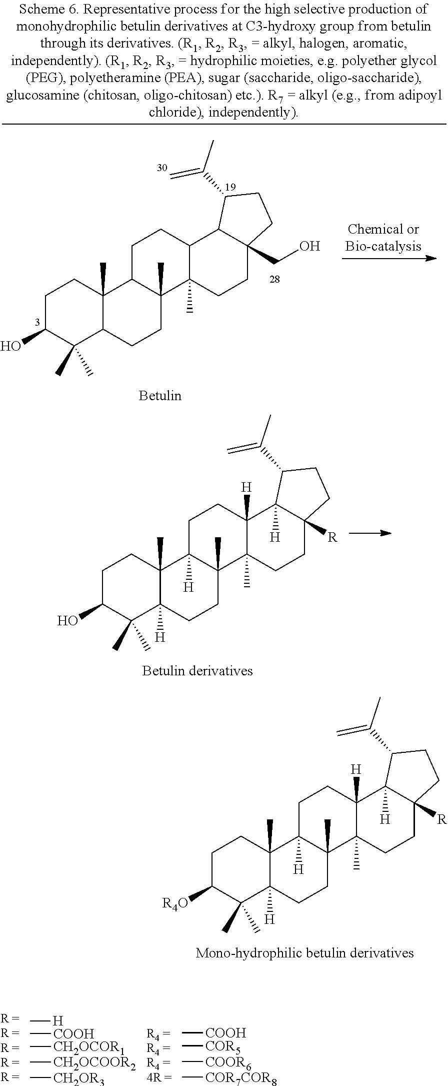

The invention relates to a process for the high selective production of mono-hydrophilic betulin derivatives at C28-hydroxy group from betulin through its derivatives (Scheme 7). The carbonated betulin can be further functionalized with hydrophilic moieties by chemical or bio-catalysis (Scheme 7). The resulting mono-hydrophilic betulin derivatives can be used as a surfactant with amphiphilic structure and properties.

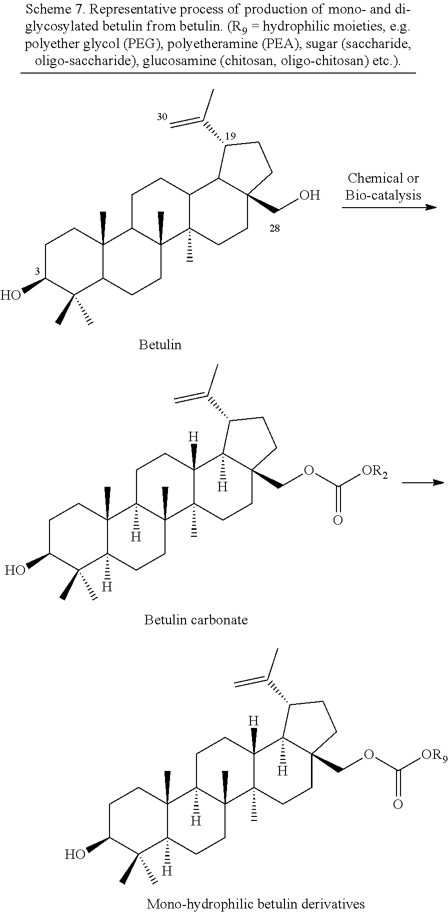

The invention relates to a process for the high selective production of di-hydrophilic betulin derivatives at C28- and C3-hydroxy group from betulin through its derivatives based on Scheme 6 and 7. In this case, R₁, R₂, R₃ in Scheme 6 can be hydrophilic moieties (polyether glycol (PEG), polyetheramine (PEA), sugar (saccharide, oligo-saccharide), glucosamine (chitosan, oligo-chitosan) etc.). The resulting di-hydrophilic betulin derivatives can be used as a surfactant with amphiphilic structure and properties.

### MODE FOR THE INVENTION

This invention is directed to the amphiphilic functional materials, mono- and di-glycosylated betulin derivatives, and related to their production method. The invention is further directed to the use of said amphiphilic functional materials, mono-, and di-glycosylated betulin and their derivatives for surfactant applications.

### Amphiphilic Functional Materials, Mono-, and Di-Hydrophilic Moiety Derivatized Betulin Derivatives

This invention provides the amphiphilic functional materials, mono-, and di-hydrophilic moiety derivatized betulin derivatives. Betulin derivatives can be betulin anhydrous (R═H) dehydrated at primary alcohol of betulin, betulinic acid (R═COOH) oxidized at primary alcohol of betulin, betulinic esters (R═CH₂OCOR₁) esterified at primary alcohol of betulin, betulinic carbonates (R═CH₂OCOOR₂) carbonated at primary alcohol of betulin, and betulinic ethers (R═CH₂OR₃) etherificated at primary alcohol of betulin (Scheme 2). R₁, and R₂, can be H, and R₁, R₂, and R₃ can be selected from C₁-C₂₀ alkyl and allyl groups, independently.

### Derivatization of a Primary Alcohol in Betulin (Intermediate)

In the betulin molecule, there are two reactive hydroxyl (alcohol) groups, which can be reactive for the derivatization. Therefore to prepare nomo-glycosylated betulin derivatives, it needs to be derivatized or protected properly at the primary hydroxyl group. Both chemical and bio-catalysis can be employed to derivatize betulin. Some examples are below, but not limited.

### 1) Selective Oxidation of Betulin to Betulinc Acid by Microorganisms or Oxidative Enzymes

The primary alcohol of betulin can be selectively oxidized (almost 100% product selectivity) to acid by oxidative microorganisms or enzymes such as Gluconobacter sp., Mycobacterium sp., and Acctobactor sp. Microbial oxidation can be performed at 10-100° C. and pH2-pH10 in aqueous condition or water-organic solvent mixture system by microorganism. Organic solvent can be selected from DMF, DMSO, pyridine, THF, chloroform, dichloromethane, hydrocarbon and cyclic hydrocarbon, alkyl esters, alcohols, ketone and their mixtures, but is not limited for the reaction. The preferred solvents are water-miscible solvents such as DMF, DMSO, pyridine, THF and alcohols (glycol) or mixtures of the same or mixtures containing said solvents. Organic solvent can preferably be used at ratio of 1 to 3000%, preferably 10-1000% to water. To improve conversion rate and to prevent product inhibition, in situ recovery of resulting product can be employed by using ion exchange resin. But, the type of used resin is not limited.

### 2) Esterification of Betulin by Chemical and Enzymatic Catalysis

The betulin can be esterified with alkoxy donors such as alcohols to corresponding ester by acid or base catalysis or enzymes. Acid catalyst can be Brønsted and Lewis acids such as hydrochloric acid, sulfuric acid, hydrofluoric acid, nitric acid, phosphoric acid, toluenesulfonic acid, polystyrene sulfonate, heteropoly acid, zeolites, silico-aluminates, sulfated zirconia, transition metal oxides, and cation exchanger. Base catalyst can be Brønsted and Lewis base such as sodium hydroxide, potassium hydroxide, sodium amide, pyridine, imidazole, DBU (1,8-Diazabicycloundec-7-ene), guanidines, TBD (1,5,7-Triazabicyclo [4.4.0]dec-5-ene), solid base, metal oxide (CaO, BaO, MgO), and anion exchanger.

Lipase catalysed esterification of the betulin has been achieved with different acyl donors such as propionic acid, ethylacetate, n-butylacetate and vinylacetate in a solvent system using immobilized Candida antarctica lipase B, Novozym®435 (N435). But acyl donor and enzyme are not limited for the reaction. The ratio of N435 is 1-300%, preferably at a ratio of 5-50% to betulin. Organic solvent can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. The preferred solvents are DMF, DMSO, pyridine, THF and alcohols (glycol) or mixtures of the same or mixtures containing said solvents. Organic solvent can preferably be used at ratio of 1 to 3000%, preferably 10-1000% to betulin. The ratio of acyl donors is 10-1000%, preferably 10-300% to betulin. Different esterase from various sources can be used for the reaction. The optimum temperature of lipase including Candida antarctica lipase B is in the literature generally reported to be around 60° C. But the optimum can vary depending on solvents and reaction times. The selectivity and yield of product by the process of the present invention can accordingly also be changed by varying the conditions.

### 3) Carbonation of Betulin by Chemical and Enzymatic Catalysis

The betulin can be carbonated with donors such as dimethylcarbonate, diethyl-carbonate and diphenylcarbonate to corresponding carbonate by acid or base catalysis or enzymes. Acid catalyst can be Brønsted and Lewis acids such as hydrochloric acid, sulfuric acid, hydrofluoric acid, nitric acid, phosphoric acid, toluenesulfonic acid, polystyrene sulfonate, heteropoly acid, zeolites, silico-aluminates, sulfated zirconia, transition metal oxides, and cation exchanger. Base catalyst can be Brønsted and Lewis base such as sodium hydroxide, potassium hydroxide, sodium amide, pyridine, imidazole, DBU (1,8-Diazabicycloundec-7-ene), guanidines, TBD (1,5,7-Triazabicyclo [4.4.0]dec-5-ene), solid base, metal oxide (CaO, BaO, MgO), and anion exchanger.

Molecular sieves mediated carbonation of the betulin can be achieved with dimethyl-carbonate, diethylcarbonate or diphenylcarbonate in a solvent system or solventless condition. The molecular sieves are not limited, but properly 4 Å-5 Å molecular sieves. The ratio of molecular sieves is 10-2000%, preferably at a ratio of 50-1000% to betulin. Organic solvent can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. The preferred solvents are DMF, DMSO, pyridine, THF and alcohols (glycol) or mixtures of the same or mixtures containing said solvents. Organic solvent can preferably be used at ratio of 1 to 3000%, preferably 10-1000% to betulin. The ratio of carbonation agent is 10-1000%, preferably 10-300% to betulin. The optimum temperature is 20-200° C., preferably 80-150° C. But the optimum can vary depending on solvent systems, ration of molecular sieves and reaction times. The selectivity and yield of product by the process of the present invention can accordingly also be changed by varying the conditions.

### Mono-Glycosylation of Betulin Derivatives (Scheme 2)

In nature, glycoside synthesis is a common reaction providing a variety of oligosac-charides and glycoconjugates as glycolipids, glycoproteins and glycopeptides. The glycosylation of betulin derivatives protected at the primary alcohol of betulin can be performed for the O-glycoside bond formation by chemical and bio-catalysis. The chemical O-glycoside bond formation of the betulin derivatives can be archived by several approaches such as Koenigs-Knorr method and trichloroacetimidate method.

In the trichloroacetimidate method, various O-glycosyl trichloroacetimidates can be used as glycosyl donors, which are easily prepared, sufficiently stable. The O-glycosyl trichloroacetimidates can be activated for the glycosylation reactions with catalytic amounts of Lewis acids. Lewis acids can be selected from TMSOTf, BF₃·Et₂O, Sn(OTf)₂, AgOTf and ZnCl₂·Et₂O, but is not limited for the reaction. O-Glycosyl trichloroacetimidates can be prepared from various sugar groups such as arabinose, glucose, mannose, and rhamnose, but is not limited for the donors. Glycosylation can be performed in solvent, which can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. Organic solvent can preferably be used at ratio of 1 to 3000%, preferably 10-1000% to betulin. The ratio of glycosyl donors to the betulin derivatives can be 0.1-10, preferably 0.5-2. The optimum temperature is-30-100° C., preferably-20-50° C. The temperature can be controlled from low to high for the reaction. But the optimum can vary depending on solvent systems, ratio of donor, and reaction times.

Several types of biocatalysts from different microorganisms can be used in the enzymatic glycosylation of betulin and its derivatives, and include glycosyltransferases and trans-glycosidases. And whole-cell biotransformation systems capable of re-generating the activated sugar cofactor, such as fungi, bacteria, and plant-cell cultures can be applied for the glycosylations.

### Mono and Di-Glycosylation of Betulin (Scheme 3)

In nature, glycoside synthesis is a common reaction providing a variety of oligosac-charides and glycoconjugates as glycolipids, glycoproteins and glycopeptides. The glycosylation of betulin can be performed for the O-glycoside bond formation by chemical and bio-catalysis. The chemical O-glycoside bond formation of betulin and betulin derivatives can be archived by several approaches such as acid-catalyzed glycosylation (etherification), Koenigs-Knorr method and trichloroacetimidate method.

In the acid-catalyzed glycosylation (etherification), primary alcohol of betulin can be etherified with sugars such as glucose, fructose and sucrose, but are not limited for the reaction. The reaction can be performed by acid catalysts such as hydrochloric acid, sulfuric acid, phosphoric acid, toluenesulfonic acid, polystyrene sulfonate, heteropoly acids, zeolites and acidic ion exchangers. In the trichloroace-timidatc method, various O-glycosyl trichloroacctimidates can be used as glycosyl donors, which are easily prepared, sufficiently stable. The O-glycosyl trichloroace-timidates can be activated for the glycosylation reactions with catalytic amounts of Lewis acids. Lewis acids can be selected from TMSOTf, BF₃·Et₂O, Sn(OTf)₂, AgOTf and ZnCl₂·Et₂O, but is not limited for the reaction. O-Glycosyl trichloroace-timidates can be prepared from various sugar groups such as arabinose, glucose, mannose, and rhamnose, but is not limited for the donors. Glycosylation can be performed in solvent, which can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. Organic solvent can preferably be used at ratio of 1 to 3000%, preferably 10-1000% to betulin. The ratio of glycosyl donors to betulin and betulin derivatives can be 0.1-20, preferably 0.5-5. The optimum temperature is-30-100° C., preferably-20-50° C. The temperature can be controlled from low to high for the reaction. But the optimum can vary depending on solvent systems, ratio of donor, and reaction times.

Several types of biocatalysts from different microorganisms can be used in the enzymatic glycosylation of betulin and its derivatives, and include glycosyltransferases and trans-glycosidases. And whole-cell biotransformation systems capable of re-generating the activated sugar cofactor, such as fungi, bacteria, and plant-cell cultures can be applied for the glycosylations.

### Mono-(Poly)Alkoxylation of Betulin Derivatives (Scheme 4)

Mono-(poly)alkoxylation can be achieved by alkoxylation of betulin derivatives in solvent using alkoxylation agents such as ethylene oxide, propylene oxide, and their mixture in present of catalyst. The catalyst can be KOH, NaOH or Phosphazene base t-BuP₄, but not limited. Organic solvent can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. The preferred solvents arc DMF, DMSO, pyridine, or THF or mixtures of the same or mixtures containing said solvents. Organic solvent can preferably be used at ratio of 1 to 3000%, preferably 10-1000% to betulin. The ratio of alkoxylation agents can be controlled to obtain a desired alkoxyl repeating units. The optimum temperature is −30-300° C., preferably 40-200° C. The temperature can be controlled from low to high for the reaction. But the optimum can vary depending on solvent systems, ration of ethylene oxide and reaction times. The size of ethoxyl chain and yield of product by the process of the present invention can accordingly also be changed by varying the conditions.

### Di-(poly)Alkoxylation of Betulin (Scheme 5)

Di-(poly)alkoxylation of betulin can be achieved by alkoxylation of betulin in solvent using using alkoxylation agents such as ethylene oxide, propylene oxide, and their mixture in present of catalyst. The catalyst can be KOH, NaOH or Phosphazene base t-BuP₄, but not limited. Organic solvent can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. The preferred solvents are DMF, DMSO, pyridine, or THF or mixtures of the same or mixtures containing said solvents. Organic solvent can preferably be used at ratio of 1 to 3000%, preferably 10-1000% to betulin. The ratio of ethylen oxide can be controlled to obtain a desired ethoxyl repeating units. The optimum temperature is −30-300° C., preferably 40-200° C. The temperature can be controlled from low to high for the reaction. But the optimum can vary depending on solvent systems, ration of ethylene oxide and reaction times. The size of ethoxyl chain and yield of product by the process of the present invention can accordingly also be changed by varying the conditions.

### HLB Values of Mono- and Di-Hydrophilic Moiety Derivatized Betulin Derivatives

HLB values of mono and di-hydrophilic moiety derivatized betulin derivatives are calculated by Grifin's method. HLB (Hydrophile-Lipophile Balance) value can vary depending on the hydrophilic moiety groups.

### Critical Micelle Concentration (CMC) Determination of Mono- and Di-HyDrophilic Moiety Derivatized Betulin Derivatives as Surfactants

CMC of mono and di-hydrophilic moiety derivatized betulin derivatives were determined according to the stalagmometric method, which is one of the most common methods for measuring surface tension. Samples were prepared at different concentration of mono and di-hydrophilic moiety derivatized betulin derivatives. The surface tension of samples was calculated from the number of drops obtained by stalagmometer. CMC was calculated by plotting surface tensions vs. concentrations of sample.

### Analysis of Reaction and Material by FT-IR and NMR

The formation of new functional groups at the primary hydroxyl group of betulin was determined using samples collected from the reaction by FT-IR analysis. The spectra of samples were obtained in region of 500-4000 cm⁻¹ using Nicolet-iS5 (Thermo Scientific, USA). An air background spectrum was collected before the analysis of the sample, and subtracted from each sample spectrum.

Quantitative analyses and product elucidation were performed using by 1H-NMR using 400 MHz NMR (Bruker, UltraShield Plus 400, Germany).

### Example

The present invention is further explained in more detail with reference to the following examples. These examples, however, should not be interpreted as limiting the scope of the present invention in any manner. In the examples, the glycosylations of betulin and betulin derivative were performed using 2,3,4,6-tetra-O-benzoyl-α-D-glucopyranosyl trichloroacetimidate and glucose as representative glycosyl donors.

### Example 1. Derivatization of Betulin at the Primary Hydroxyl Group in Dimethyl-Carbonate in Present of Molecular Sieves (Scheme 8)

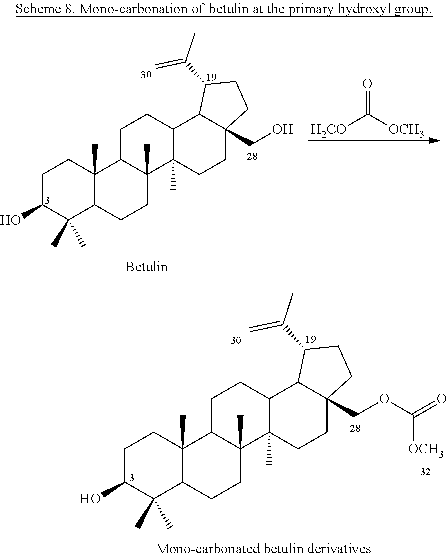

300 mg betulin and 1.5 g molecular sieves (4 Å) were added in 5 mL dimethyl-carbonate in a 10 mL reaction vessel. The reaction was performed with increasing temperature up to 110° C. for 2 hr. 98% conversion of betulin was observed in the reaction. And 80.5% selectivity and 78.9% yield (237 mg, w/w) of mono-carbonated betulin was obtained after simple recovery by filtration and evaporation of residual dimethylcarbonate. The carbonation at the primary hydroxyl group was confirmed by FT-IR and NMR. Strong peak of carbonyl (C═O) was appeared at 1746 cm⁻¹ in FT-IR after carbonation of betulin.

Betulin; ¹H NMR (400 MHZ, CDCl₃), δ (ppm)=4.708 & 4.606 (30CH₂, 2H, dd), 3.815 & 3.355 (28CH₂, 2H, dd), 3.209 (3CH, 1H, m), 2.402 (19CH, 1H, m), 0.5˜2.1 (others, m).

Mono-carbonated betulin; ¹H NMR (400 MHZ, CDCl₃), δ (ppm)=4.715 & 4.615 (30CH₂, 1H,d), 4.367 & 3.941 (28CH₂, 2H, dd), 3.790 (32CH₃, 3H, s), 3.206 (3CH, 1H, m), 2.448 (19CH, 1H, m), 0.5˜2.1 (others, m).

### Example 2. Mono-glycosylation of Carbonated Betulin Using 2,3,4,6-tetra-O-benzoyl-R-D-glucopyranosyl trichloroacetimidate (Scheme 9)

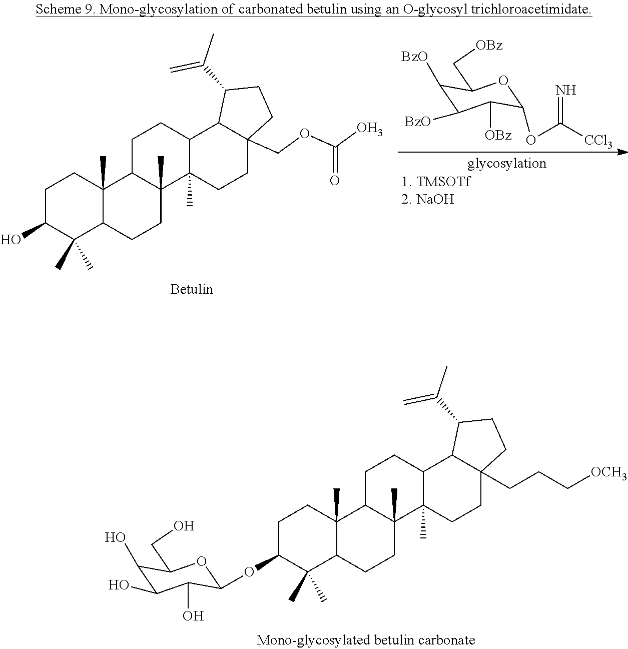

The resulting carbonated betulin was subjected to glycosylation. 50 mg carbonated betulin (0.1 mmol) was dissolved in 2 mL anhydrous dichloromethane in 10 mL reaction vessel, and the solution was stirred with 4 Å molecular sieves at −10° C. for 60 min.

A catalyst, TMSOTf (0.02 mmol) was added under argon, followed by dropwise addition of donor solution, 2,3,4,6-tetra-O-benzoyl-α-D-glucopyranosyl trichloroacetimidate (74.1 mg, 0.1 mmol) in 2 mL anhydrous dichloromethane for 10 min under stirring. The glycosylation reaction temperature was gradually increased to room temperature for 5 hr, and the reaction was quenched by addition of trimethylamine (0.1 mL, 0.75 mmol). After evaporation of the solvent, the resulting residue dissolved in a mixture of methanol/THF/H2O (1/2/1, 4 mL) was added NaOH (2.0 mmol). The mixture was stirred overnight at room temperature and then acidified to pH 4 with aqueous 10% HCl. After evaporation of the solvents, the solid residue was purified by reversed-phase flash chromatography (MeOH/H2O, 7:3 to 9:1). 56.5 mg (95% purity, 81% mol/mol yield) glucose-betulin carbonate was obtained.

The glycosylation was confirmed as peak shift and change of ¹H NMR (400 MHZ, CDCl₃) from δ (ppm)=3.209 (3CH, 1H, m) of carbonated betulin to 3.407 (3CH, 1H, dd) of glucose-betulin carbonate. Molecular weight of product was measured as m/z 662.95 [M]⁺ (calculated for C₃₈H₆₂O₉, 662.89).

### Example 3. Di-glycosylation of Betulin Using 2,3,4,6-tetra-O-benzoyl-R-D-glucopyranosyl trichloroacetimidate (Scheme 10)

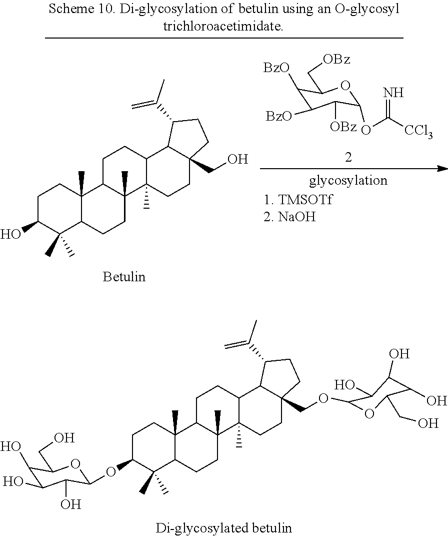

44.2 mg betulin (0.1 mmol) was dissolved in 2 mL anhydrous dichloromethane in 10 mL reaction vessel, and the solution was stirred with 4 Å molecular sieves at −10° C. for 60 min. A catalyst, TMSOTf (0.02 mmol) was added under argon, followed by dropwise addition of donor solution, 2,3,4,6-tetra-O-benzoyl-α-D-glucopyranosyl trichloroacetimidate (148.2 mg, 0.2 mmol) in 2 mL anhydrous dichloromethane for 10 min under stirring. The glycosylation reaction temperature was gradually increased to room temperature for 5 hr, and the reaction was quenched by addition of trimethylamine (0.1 mL, 0.75 mmol). After evaporation of the solvent, the resulting residue dissolved in a mixture of methanol/THF/H2O (1/2/1, 4 mL) was added NaOH (2.0 mmol). The mixture was stirred overnight at room temperature and then acidified to pH 4 with aqueous 10% HCl. After evaporation of the solvents, the solid residue was purified by reversed-phase flash chromatography (MeOH/H2O, 7:3 to 9:1). 60.7 mg (94.5% purity, 75% mol/mol yield) diglucose-betulin was obtained.

The glycosylation was confirmed as peak shift and change of ¹H NMR (400 MHZ, CDCl₃) from δ (ppm)=3.209 (3CH, 1H, m) of betulin to 3.407 (3CH, 1H, dd) of glucose-betulin, and δ (ppm)=3.815 & 3.355 (28CH₂, 2H, dd) of betulin to 4.102 (28CH, 2H, d) of glucose-betulin. Molecular weight of product was measured as m/z 767.14 [M]⁺ (calculated for C₄₂H₇₀O₁₂, 767.00).

### Example 4. Mono-glycosylation of betulin by acid catalytic etherification (Scheme 11)

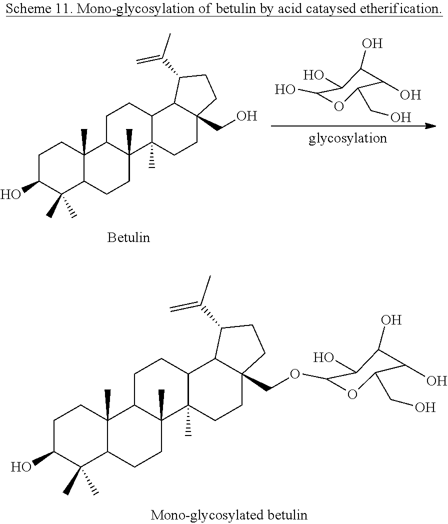

44.2 mg betulin (0.1 mmol) and 21.6 mg glucose (0.12 mmol) were dissolved in 2 mL anhydrous DMSO in 10 mL reaction vessel, and the solution was stirred with 4 Å molecular sieves at 100° C. for 10 minute. A catalyst, p-toluenesulfonic acid (0.02 mmol) was added under argon, followed by heating at 120° C. for 60 minute.

After evaporation of the solvents under reduced pressure, the solid residue was purified by reversed-phase flash chromatography (MeOH/H2O, 7:3 to 9:1). 33.1 mg (96.5% purity, 55% mol/mol yield) glucose-betulin was obtained.

The glycosylation was confirmed as peak shift and change of ¹H NMR (400 MHZ, CDCl₃) from δ (ppm)=3.815 & 3.355 (28CH₂, 2H, dd) of betulin to 4.102 (28CH, 2H, d) of glucose-betulin. Molecular weight of product was measured as m/z 604.98 [M]⁺ (calculated for C₃₆H₆₀O₇, 604.86).

### Example 5. Mono-(poly) ethoxylation of carbonated betulin using ethylene oxide (Scheme 12)

The resulting carbonated betulin was subjected to ethoxylation. 50 mg carbonated betulin (0.1 mmol) was dissolved in 2 mL THF in 10 mL reaction vessel, and 0.05 mL phosphazene base t-BuP₄ solution was added. Then the reaction vessel was cooled down to −20° C. in NaCl-ice bath, followed by gradually addition of 0.75 mL ethylene oxide solution (about 3M in THF). The ratio of betulin (0.1 mmol) and ethylene oxide (2.25 mmol) was 1 to 22.5. The reaction temperature was gradually raised to 45° C., and maintained for 48 hr. After quenching the reaction with 1 mL acetic acid, the ethoxylated betulin derivative was isolated by filtration of cation exchange resin treated to remove catalyst and evaporation of THF. The ethoxylation was confirmed as strong peak at δ (ppm)=3.6˜3.8 for ethoxylated group (OCH₂) was added in ¹H NMR of the carbonated betulin (400 MHZ, CDCl₃). Molecular weight of product was estimated as about average 1450 g/mol.

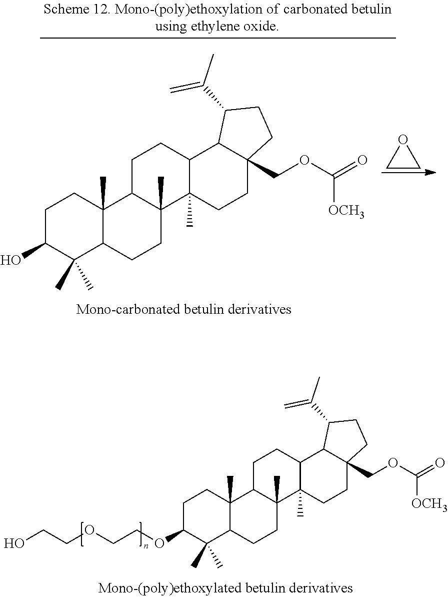

### Example 6. Di-(poly) ethoxylation of betulin using ethylene oxide (Scheme 13)

44.2 mg betulin (0.1 mmol) was dissolved in 2 mL THF in 10 mL reaction vessel, and 0.05 mL phosphazene base t-BuP₄ solution was added. Then the reaction vessel was cooled down to −20° C. in NaCl-ice bath, followed by gradually addition of 1.5 mL ethylene oxide solution (about 3M in THF). The ratio of betulin (0.1 mmol) and ethylene oxide (4.5 mmol) was 1 to 45 (1 to 22.5 for hydroxyl group to ethylene oxide). The reaction temperature was gradually raised to 45° C., and maintained for 48 hr. After quenching the reaction with 1 mL acetic acid, ethoxylated betulin derivatives was isolated by filtration of cation exchange resin treated to remove catalyst and evaporation of THF. The ethoxylation was confirmed as strong peak at & (ppm)=3.6˜3.8 for ethoxylated group (OCH₂) was added in ¹H NMR of the betulin (400 MHZ, CDCl₃). Molecular weight of product was estimated as about average 2200 g/mol.

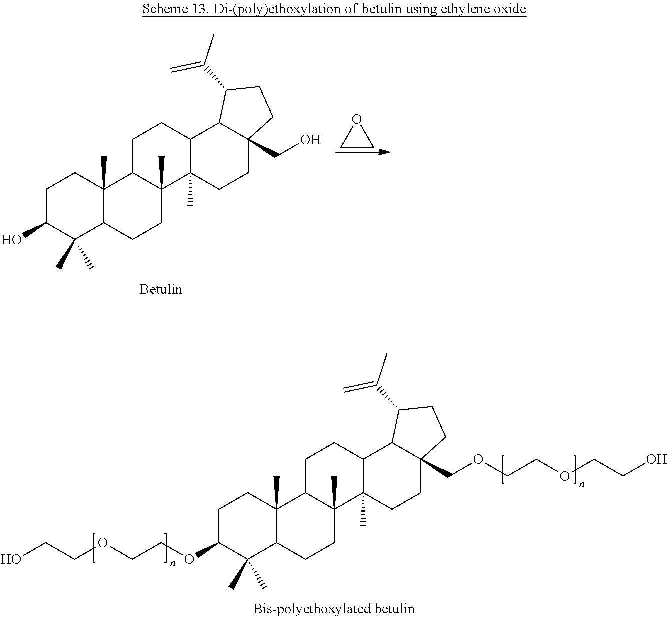

### Example 7. HLB Value

HLB values of resulting products were estimated by Grifin's method. Based on the portion of hydrophilic group and molecular weight, 5.4, 5.6 and 9.4 of HLB values were obtained from mono-glycosylated betulin carbonate, mono-glycosylated betulin and di-glycosylated betulin, respectively. Thus the hydrophobic (oil soluble-water dispersible) property is expected from the range of HLB values. And the higher HLB range can be obtained by using oligo-saccharide O-glycosyl trichloroacetimidates as glycosyl donors.

### Example 8. Critical Micelle Concentration (CMC)

CMCs of mono-glycosylated betulin, di-glycosylated betulin, mono-(poly) ethoxylated-carbonated betulin and di-(poly) ethoxylated betulin were determined in water, respectively. Surface tensions of samples obtained in above examples were determined at different concentrations according to the stalagmometric method. And CMC was obtained as 0.8 mM (mono-glycosylated betulin), 1.0 mM (di-glycosylated betulin), 0.6 mM (mono-(poly) ethoxylated-carbonated betulin) and 0.7 mM (di-(poly) ethoxylated betulin), respectively, by plotting surface tensions vs. concentrations of sample. These are comparable to ordinary non-ionic surfactants, and can be improved by increasing hydrophilicity in hydrophilic moiety.

This invention is directed to the amphiphilic functional materials, mono-hydrophilic betulin derivatives, and related to their production method. The invention is further directed to the use of said amphiphilic functional materials, mono-hydrophilic betulin and their derivatives for surfactant applications.

### Betulin Derivatives (First Intermediate)

This invention provides the amphiphilic functional materials, mono-hydrophilic moiety derivatized betulin derivatives.

1) Betulin derivatives can be betulin anhydrous (R═H) dehydrated at primary alcohol of betulin, betulinic acid (R═COOH) oxidized at primary alcohol of betulin, betulinic esters (R═CH₂OCOR₁) esterified at primary alcohol of betulin, betulinic carbonates (R═CH₂OCOOR₂) carbonated at primary alcohol of betulin, and betulinic ethers (R═CH₂OR₃) etherificated at primary alcohol of betulin (Scheme 6). R₁, and R₂, can be H, and R₁, R₂, and R₃ can be selected from C₁-C₂₀ alkyl and allyl groups, independently.

2) Carbonation of a primary alcohol in betulin (Scheme 6) as an example. In the betulin molecule, there are two reactive hydroxyl (alcohol) groups, which can be reactive for the derivatization. Therefore to prepare nomo-hydrophilic betulin derivatives, it needs to be derivatized or protected properly at the primary hydroxyl group. Both chemical and bio-catalysis can be employed to derivatize betulin. Some examples are below, but not limited. Molecular sieves mediated carbonation of the betulin can be achieved with dimethylcarbonate, diethylcarbonate or diphenylcarbonate in a solvent system or solventless condition. The molecular sieves are not limited, but properly 4 Å-5 Å molecular sieves. The ratio of molecular sieves is 10-2000%, preferably at a ratio of 50-1000% to betulin. Organic solvent can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. The preferred solvents are DMF, DMSO, pyridine, THF and alcohols (glycol) or mixtures of the same or mixtures containing said solvents. Organic solvent can preferably be used at ratio of 1 to 3000%, preferably 10-1000% to betulin. The ratio of carbonation agent is 10-1000%, preferably 10-300% to betulin. The optimum temperature is 20-200° C., preferably 80-150° C. But the optimum can vary depending on solvent systems, ration of molecular sieves and reaction times. The selectivity and yield of product by the process of the present invention can accordingly also be changed by varying the conditions.

### Preparation of Amphiphilic Functional Materials, Mono-Hydrophilic Moiety Derivatized Betulin Derivatives at C₃-Hydroxy Group of Betulin.

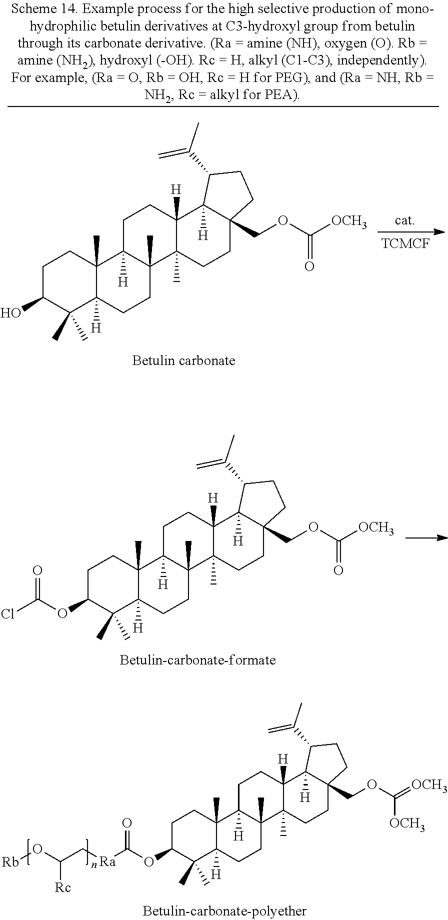

Scheme 14 describes more detail description from Scheme 6 as an example. C3-hydroxyl group of betulin carbonate (first intermediate) can be activated to chloroformate by reaction with e.g. trichloromethyl chloroformate in e.g.

N,N-dimethylaniline in solvent. The reaction can be performed in solvent, which can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. Organic solvent can preferably be used at ratio of 1 to 3000%, preferably 10-1000% to betulin. The ratio of chloroformate donor to the betulin carbonate can be 0.1-10, preferably 0.5-2. The ratio of N,N-dimethylaniline or other base substance to the betulin carbonate can be 0.1-10, preferably 0.5-2.

The resulting betulin-carbonate-formate (second intermediate) can be further functionalized with hydrophilic moieties such as PEG, PEA, sugar (saccharide, oligo-saccharide), glucosamine (chitosan, oligo-chitosan), etc. For example, a PEA, jeffamine ED-600 as a hydrophilic moiety can react with chloroformate of betulin carbonate. The reaction can be performed without solvent or in solvent, which can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. Organic solvent can preferably be used at ratio of 1 to 3000%, preferably 10-1000% to betulin-carbonate-formate. The ratio of hydrophilic moiety to the betulin-carbonate-formate can be 0.1-10, preferably 1-5. The optimum temperature is-30-100° C., preferably-20-50° C. The temperature can be controlled from low to high for the reaction. But the optimum can vary depending on solvent systems, ratio of hydrophilic moieties, and reaction times. The reaction can be performed by bio- or chemical catalysis.

The size (molecular weight) of hydrophilic moieties in the process of the present invention can also be changed by using different size of hydrophilic donors. For example, the average molecular weight of PEG can be 400, 3000, 10,000 or 50,000. For example, the average molecular weight of PEA can be 400, 3000, 10,000 or 50,000. For example of PEA can be jeffamine ED-600, jeffamine ED-900 from Croda.

### Preparation of Amphiphilic Functional Materials, Mono-Hydrophilic Moiety Derivatized Betulin Derivatives at C28-Hydroxy Group of Betulin.

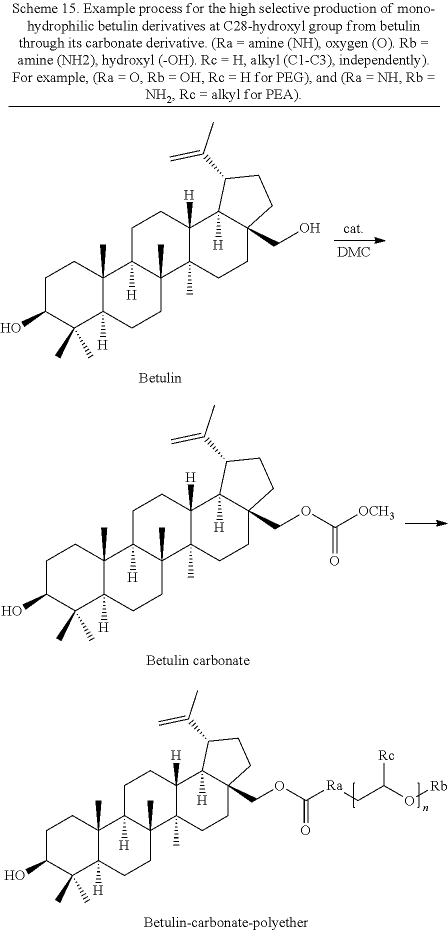

Scheme 15 describes more detail description from Scheme 7 as an example. Betulin carbonate can be further functionalized with hydrophilic moieties such as PEG, PEA, sugar (saccharide, oligo-saccharide), glucosamine (chitosan, oligo-chitosan), etc. For example, a PEG400 as a hydrophilic moiety can react with carbonate of betulin carbonate.

The reaction can be performed without solvent or in solvent, which can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. Organic solvent can preferably be used at ratio of 1 to 3000%, preferably 10-1000% to betulin carbonate. The ratio of hydrophilic moiety to the betulin carbonate can be 0.1-10, preferably 1-5. The optimum temperature is-30-250° C., preferably 50-150° C. The temperature can be controlled from low to high for the reaction. But the optimum can vary depending on solvent systems, ratio of hydrophilic moieties, and reaction times. The reaction can be performed by bio- or chemical catalysis. Catalysts can be selected from Titanium (IV) butoxide, Stannous octoate, 1,8-Diazabicyclo [5.4.0]undec-7-ene, Triazabicyclodecene, NaOH, and K₂CO₃, but not limited.

The size (molecular weight) of hydrophilic moieties in the process of the present invention can also be changed by using different size of hydrophilic donors. For example, the average molecular weight of PEG can be 400, 3000, 10,000 or 50,000.

For example, the average molecular weight of PEA can be 400, 3000, 10,000 or 50,000. For example of PEA can be jeffamine ED-600, jeffamine ED-900 from Croda.

### Preparation of Amphiphilic Functional Materials, Di-Hydrophilic Moiety Derivatized Betulin Derivatives at C28- and C3-Hydroxy Group of Betulin.

C3-hydroxyl group of betulin carbonate can be activated to chloroformate by reaction with e.g. trichloromethyl chloroformate in e.g. N,N-dimethylaniline in solvent. The reaction can be performed in solvent, which can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. Organic solvent can preferably be used at ratio of 1 to 3000%, preferably 10-1000% to betulin. The ratio of chloroformate donor to the betulin carbonate can be 0.1-10, preferably 0.5-2. The ratio of N,N-dimethylaniline or other base substance to the betulin carbonate can be 0.1-10, preferably 0.5-2.

The resulting betulin-carbonate-formate can be further functionalized with hydrophilic moieties such as PEG, PEA, sugar (saccharide, oligo-saccharide), glucosamine (chitosan, oligo-chitosan), etc. For example, a PEA, jeffamine ED-600 as a hydrophilic moiety can react with chloroformate of betulin carbonate. The reaction can be performed without solvent or in solvent, which can be selected from DMF, DMSO, pyridine, THF, chloroform, dichloromethane, toluene, hydrocarbon and cyclic hydrocarbon, alkyl esters, alcohols, ketone and their mixtures, but is not limited for the reaction. Organic solvent can preferably be used at ratio of 1 to 3000%, preferably 10-1000% to betulin-carbonate-formate. The ratio of hydrophilic moiety to the betulin-carbonate-formate can be 0.1-10, preferably 1-5. The optimum temperature is-30-100° C., preferably-20-50° C. The temperature can be controlled from low to high for the reaction. But the optimum can vary depending on solvent systems, ratio of hydrophilic moieties, and reaction times. The reaction can be performed by bio- or chemical catalysis.

The resulting betulin-carbonate-polyether (PEA) can be further functionalized at C28-hydroxy (carbonate) group with another hydrophilic moieties such as PEG, PEA, sugar (saccharide, oligo-saccharide), glucosamine (chitosan, oligo-chitosan), etc.

The size (molecular weight) of hydrophilic moieties in the process of the present invention can also be changed by using different size of hydrophilic donors. For example, the average molecular weight of PEG can be 400, 3000, 10,000 or 50,000. For example, the average molecular weight of PEA can be 400, 3000, 10,000 or 50,000. For example of PEA can be jeffamine ED-600, jeffamine ED-900 from Croda.

### HLB Values of Mono- and Di-Hydrophilic Moiety Derivatized Betulin Derivatives

HLB values of mono and di-hydrophilic moiety derivatized betulin derivatives are calculated by Grifin's method. HLB (Hydrophile-Lipophile Balance) value can vary depending on the hydrophilic moiety groups.

### Critical Micelle Concentration (CMC) Determination of Mono-Hydrophilic Moiety Derivatized Betulin Derivatives as Surfactants

CMC of mono-hydrophilic moiety derivatized betulin derivatives were determined according to the stalagmometric method, which is one of the most common methods for measuring surface tension. Samples were prepared at different concentration of mono-hydrophilic moiety derivatized betulin derivatives. The surface tension of samples was calculated from the number of drops obtained by stalagmometer. CMC was calculated by plotting surface tensions vs. concentrations of sample.

### Analysis of Reaction and Material by FT-IR and NMR

The formation of new functional groups such as carbonate, amide, and chloroformate at the primary- and secondary hydroxyl group of betulin was determined using samples collected from the reaction by FT-IR analysis. The spectra of samples were obtained in region of 500-4000 cm⁻¹ using Nicolet-iS5 (Thermo Scientific, USA). An air background spectrum was collected before the analysis of the sample, and subtracted from each sample spectrum.

Quantitative analyses and product elucidation were performed using by 1H-NMR using 400 MHz NMR (Bruker, UltraShield Plus 400, Germany).

### Example

The present invention is further explained in more detail with reference to the following examples. These examples, however, should not be interpreted as limiting the scope of the present invention in any manner.

In the examples, the hydrophilic functionalizations of betulin derivative were performed using PEO (average molecular weight 400) and PEA (average molecular weight 600, Jeffamine ED-600) as representative hydrophilic moieties.

### Example 9. Derivatization of Betulin at the Primary Hydroxyl Group in Dimethyl-Carbonate in Present of Molecular Sieves (Scheme 16)

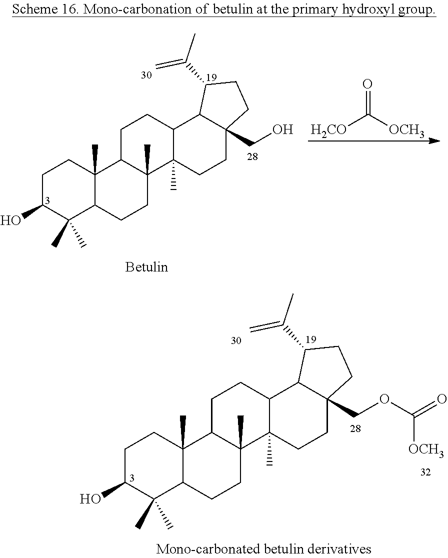

3 g betulin and 15 g molecular sieves (4 Å) were added in 50 mL dimethylcarbonate in a 250 mL reaction vessel. The reaction was performed with increasing temperature up to 110° C. for 2 hr. 98% conversion of betulin was observed in the reaction. And 80.5% selectivity and 78.9% yield (237 mg, w/w) of mono-carbonated betulin was obtained after simple recovery by filtration and evaporation of residual dimethylcarbonate. The carbonation at the primary hydroxyl group was confirmed by FT-IR and NMR. Strong peak of carbonyl (C═O) was appeared at 1746 cm⁻¹ in FT-IR after carbonation of betulin.

Betulin; ¹H NMR (400 MHZ, CDCl₃), δ (ppm)=4.708 & 4.606 (30CH₂, 2H, dd), 3.815 & 3.355 (28CH₂, 2H, dd), 3.209 (3CH, 1H, m), 2.402 (19CH, 1H, m), 0.5˜2.1 (others, m).

Mono-carbonated betulin; ¹H NMR (400 MHZ, CDCl₃), δ (ppm)=4.715 & 4.615 (30CH₂, 1H,d), 4.367 & 3.941 (28CH₂, 2H, dd), 3.790 (32CH₃, 3H, s), 3.206 (3CH, 1H, m), 2.448 (19CH, 1H, m), 0.5˜2.1 (others, m).

### Example 10. Mono-Hydrophilic Functionalization at C3-Hydroxyl Group of Carbonated Betulin Using PEA (Jeffamine ED-600) (Scheme 14, 17)

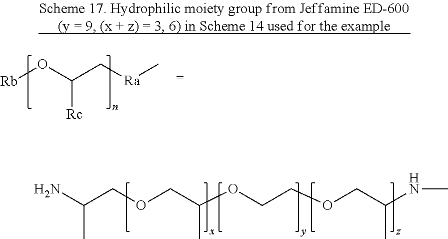

The resulting Betulin-carbonate (first intermediate) obtained in example 9 was subjected to further functionalization using hydrophilic moiety (Jeffamine ED-600, Scheme 7). C3-hydroxyl group of betulin carbonate were activated to chloroformate by reaction with trichloromethyl chloroformate (TCMCF) in N,N-dimethylaniline (DMA) in solvent. 100 mg Betulin-carbonate (0.2 mmol) was dissolved in 0.5 mL anhydrous THF in 4 mL reaction vessel at 0° C., and followed by addition of 47.5 mg (0.24 mmol) TCMCF and 29 mg (0.24 mmol) DMA in 0.5 mL anhydrous THF. The solution was stirred at room temperature for 12 hr. After reaction completed, diethylether was added in the solution, followed by washing with 1 mL 0.2M HCl, 1 mL 0.2M NaOH, and 1 mL deionized water, consequently. After drying using Na₂SO₄ and removal of solvent, white powder product (Betulin-carbonate-formate) was quantitatively obtained (105 mg), and used for further functionalization.

The resulting 50 mg Betulin-carbonate-formate (second intermediate) was added to 200 mg Jeffamine ED-600 in 4 mL vial, and mixed at room temperature for 30 min. To remove Jeffamine ED-600 remained in the reaction, 1 mL diethylether and 1 mL deionized water were added, and product was extracted 3 times into diethylether phase. After washing using deionized water and removal of solvent, 55 mg amphiphilic product (Betulin-carbonate-polyether (PEA)) was obtained.

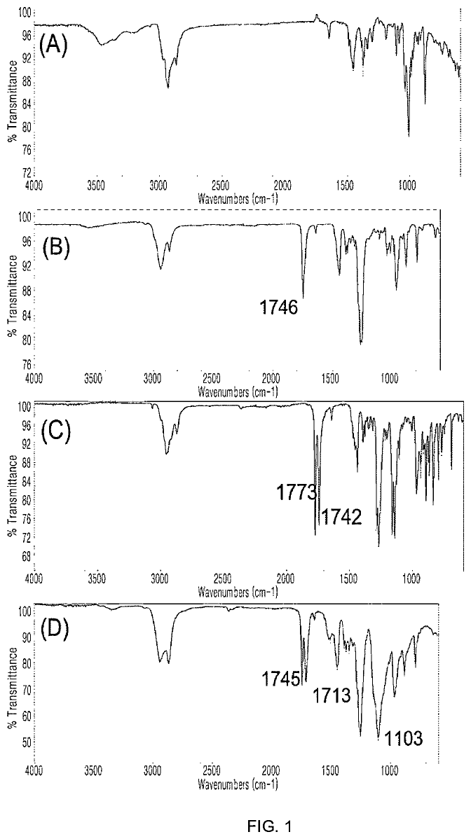

The functional group changes in the individual steps were analysed by FT-IR. A strong carbonate-carbonyl peak was appeared at 1746 cm⁻¹ in Betulin-carbonate (FIG. 1B) after carbonation of betulin (FIG. 1A). Then another strong formate-carbonyl peak were added at 1773 cm⁻¹ in Betulin-carbonate-formate (FIG. 1C) after activation of C3-hydroxyl group of Betulin-carbonate (FIG. 1B). In the further functionalization using Jeffamine ED-600, the formate-carbonyl peak was disappeared, and a new carbonate-carbonyl peak and ether peak were appeared at 1713 cm⁻¹ and 1103 cm⁻¹, respectively, in the product (Betulin-carbonate-polyether (PEA)) (FIG. 1D).

The final chemical structure prepared according to the example 10 is as follows.

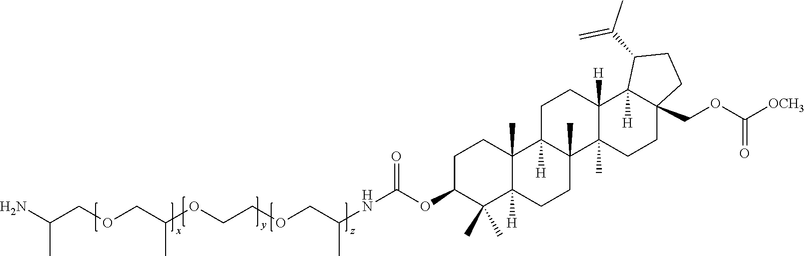

wherein y=9 and (x+z)=3.6

### Example 11. Mono-Hydrophilic Functionalization at C28-Hydroxyl Group of Betulin Using PEG (PEG400) Through Mono-Carbonation of Betulin (Scheme 15, 18)

The resulting Betulin-carbonate obtained in example 1 was subjected to further functionalization using hydrophilic moiety PEG400 (Scheme 15). The carbonate functional group at C28-hydroxyl of betulin can be reacted with primary alcohol group of hydrophilic PEG400 (Scheme 18). The resulting 50 mg Betulin-carbonate in 1 mL toluene was added to 200 mg PEG400 in 4 mL vial, and followed by addition of catalyst, 25 mg stannous octoate. The reaction was performed at 125° C. for 12 hr.

To remove PEG400 remained in the reaction, 1 mL diethylether and 1 mL deionized water were added, and product was extracted 3 times into diethylether phase. After washing using deionized water and removal of solvent, 65 mg amphiphilic product (Betulin-carbonate-polyether (PEG)) was obtained.

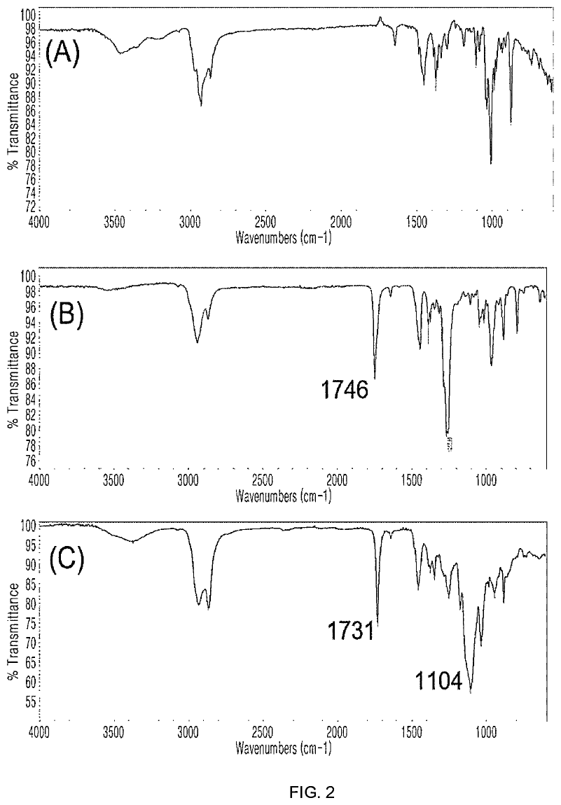

The functional group changes in the individual steps were analysed by FT-IR. A strong carbonate-carbonyl peak of was appeared at 1746 cm⁻¹ in Betulin-carbonate (FIG. 2B) after carbonation of betulin (FIG. 2A). In the further functionalization using PEG400, the carbonate-carbonyl peak was shifted to 1731 cm⁻¹, and a new ether peak was appeared at 1104 cm⁻¹ in the product (Betulin-carbonate-polyether (PEG)) (FIG. 2C).

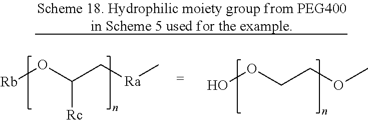

The final chemical structure prepared according to the example 11 is as follows.

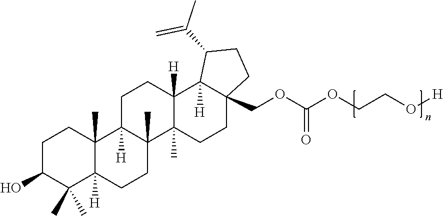

Wherein, n=8.2˜9.1

### Example 12. HLB Value

HLB values of resulting products were estimated by Grifin's method. Based on the portion of hydrophilic group and molecular weight. 10.9 and 8.9 of HLB values were obtained from Betulin-carbonate-polyether (PEA) from Example 10, and Betulin-carbonate-polyether (PEG) from Example 11, respectively. Thus, they can be used as O/W (oil in water) emulsifying agent expected from the range of HLB values.

### Example 13. Critical Micelle Concentration (CMC)

CMCs of two mono-hydrophilic functionalized betulin at C28-hydroxy and C3-hydroxy group were determined in water, respectively. Surface tensions of samples obtained in above examples were determined at different concentrations according to the stalagmometric method. And CMC was obtained as 0.6 mM (Betulin-carbonate-polyether (PEA) from Example 10), and 0.5 mM (Betulin-carbonate-polyether (PEG) from Example 11), respectively, by plotting surface tensions vs. concentrations of sample. These are comparable to ordinary non-ionic surfactants, and can be improved by increasing hydrophilicity in hydrophilic moiety.

### Example 14. Cosmetic (Lotion) Formulation

The formulations were performed at the certain contents as shown in Table 1 below. Samples 1 to 4 in Table 1 below are obtained using Example 10 ((Betulin-carbonate-polyether (PEA), BT-PEA) or Example 11 (Betulin-carbonate-polyether (PEG), BT-PEG).

The Oil and aqeous phase were separatly prepared at 40-70° C., and mixed using me-chanical mixer for emulsifying. After cooling to room temperature, the emulsified solutions were compared with control, apparently.

The stability of emulsified solution was compared with control after 1 month storage at 20° C.

As a result, it was confirmed that the Samples 1 to 4 using the examples of the present invention as surfactants exhibited stability equivalent to that of the control.

TABLE 1

| | | Content (%) | | | | |
|---|---|---|---|---|---|---|
| Phase | Ingradients | Control | Sample 1 | Sample 2 | Sample 3 | Sample 4 |
| Oil | Cetostearyl alcohol | 0.50 | 0.50 | 0.50 | 0.50 | 0.50 |
| | Squalane | 5.00 | 5.00 | 5.00 | 5.00 | 5.00 |
| | Ethylhexylglycerine | 3.10 | 3.10 | 3.10 | 3.10 | 3.10 |
| | Phenoxyethanol | 0.20 | 0.20 | 0.20 | 0.20 | 0.20 |
| | Dimethicone | 0.30 | 0.30 | 0.30 | 0.30 | 0.30 |
| | Glyceryl stearate | 1.00 | 1.00 | 1.00 | | 1.00 |
| | Glycerylstearate/ | 1.00 | | | | |
| | PEG-100 stearate | | | | | |
| | PEG-Stearate | 1.00 | 1.00 | | | |
| | Sorbitan stearate | 0.50 | 0.50 | | | |
| | BT-PEG(Example 11) | | 1.00 | 2.50 | 3.50 | |
| | BT-PEA (Example 10) | | | | | 2.50 |
| Aqueous | Water | 63.85 | 63.85 | 63.85 | 63.85 | 63.85 |
| | Glyceine | 5.00 | 5.00 | 5.00 | 5.00 | 5.00 |
| | 1,3-Butylene glycol | 5.00 | 5.00 | 5.00 | 5.00 | 5.00 |
| | Ethylhexylglycerine | 0.20 | 0.20 | 0.20 | 0.20 | 0.20 |
| | Triethanol amine | 1.20 | 1.20 | 1.20 | 1.20 | 1.20 |
| | Cabomer | 12.00 | 12.00 | 12.00 | 12.00 | 12.00 |
| | Fragrance | 0.15 | 0.15 | 0.15 | 0.15 | 0.15 |
| Results | Formulation | emulsfied | emulsfied | emulsfied | emulsfied | emulsfied |
| | Stability after | stable | stable | stable | stable | stable |
| | 1 month | | | | | |

### INDUSTRIAL APPLICABILITY

This invention is related to amphiphilic betulin derivatives, and their efficient production method. And resulting materials can be used as biobased surfactants.

1. A compound having formula (1): 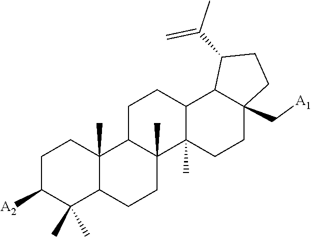
wherein,
A₁ and A₂ is independently W₁ or W₂;
W₁ is 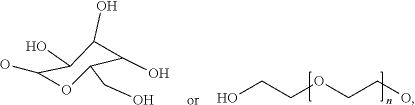
n is an integer of 1 to 100,
when one of A₁ or A₂ is W₁ and the other one is independently selected from H, (═O) OH, —OCOR₁, —OCOOR₂, —OR₃, and —OH,
R₁ and R₂ is H; or
R₁, R₂ and R₃ is independently selected from C₁-C₂₀ alkyl, allyl groups and halide group,
W₂ is 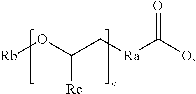
Ra is amine (NH) or oxygen (O),
Rb is amine (NH₂) or hydroxyl (—OH),
Rc is H, or C₁-C₃ alkyl,
n is an integer of 1 to 100,
when one of B₁ or B₂ is W₂, the other one is independently selected from H, (═O) OH, —OCOR₁, —OCOOR₂, —OR₃, and —OH, and
R₁, R₂ and R₃ is independently is selected from C₁-C₂₀ alkyl, allyl groups and halide group.

2. The compound of claim 1, wherein the compound of formula (1) is a betulin derivative of formula (1-1), (1-2), (1-3), (1-4), (2-1), (2-2), (2-3), or (2-4): 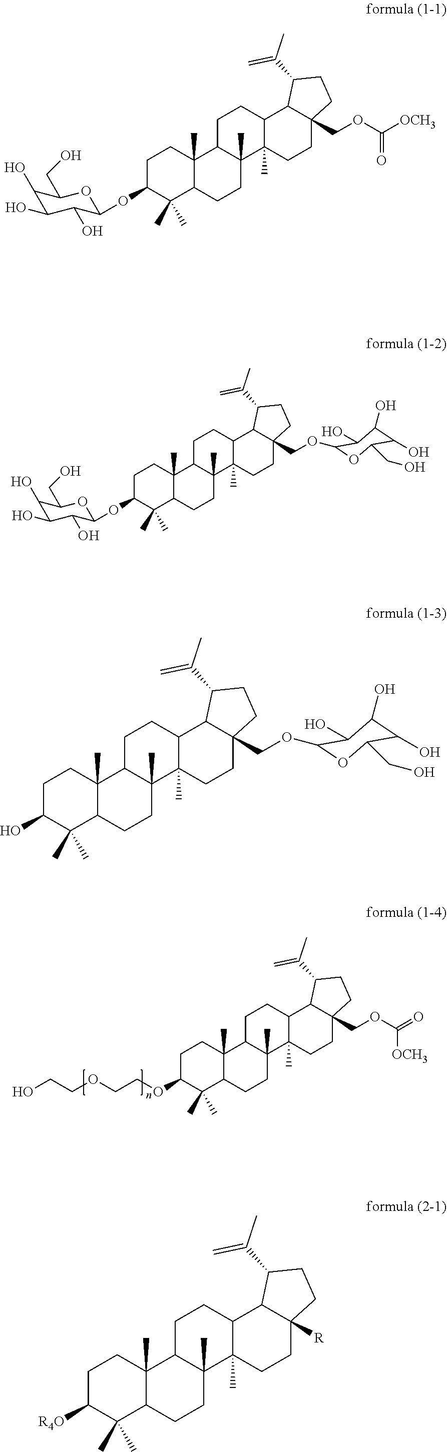
wherein, R₄ is independently selected from —COOH, —COR₅, —COOR₆, and —COR₇COR₈,
R₅, R₆ and R₈ is independently hydrophilic moieties, R₇ is alkyl.
formula (2-2) 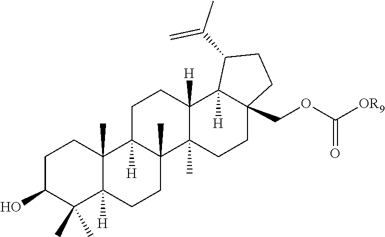
Wherein, R₉ is hydrophilic moieties, the hydrophilic moieties is selected from polyether glycol (PEG), polyetheramine (PEA), sugar, saccharide, oligo-saccharide, glucosamine, chitosan, and oligo-chitosan. 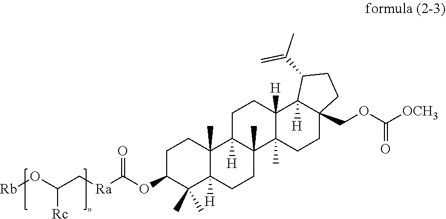
Wherein,
Ra is O, Rb is OH or H, Rc is H or CH₃ for hydrophilic moieties; or
Ra is NH, Rb is NH₂, Rc is alkyl for PEA 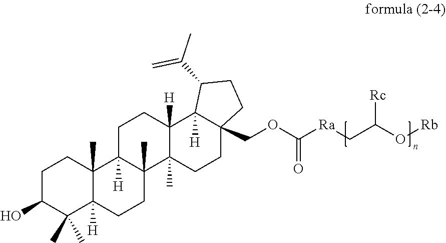
Wherein,
Ra is O, Rb is OH or H, Rc is H or CH₃ for hydrophilic moieties; or
Ra is NH, Rb is NH₂, Rc is alkyl for PEA.

3-4. (canceled)

5. A method for manufacturing a compound, the method comprising: preparing a betulin; and functionalizing the betulin.

6. The method of claim 5, wherein the functionalizing of the betulin is glycosylation or alkoxylation of the betulin, and further comprising preparing an intermediate from the betulin; and glycosylation or alkoxylation of the intermediate.

7. The method of claim 6, wherein the preparing the intermediate comprises derivatizing the betulin with acid, ester, carbonate or ether group at a primary hydroxyl group of betulin by oxidation, esterification, carbonation or etherification.

8. The method of claim 5, wherein the glycosylation is performed with glycosyl donors by chemical or bio-catalysis.

9. The method of claim 5, wherein the alkoxylation is performed with alkoxylation agents, wherein the alkoxylation agents is selected from among ethylene oxide, propylene oxide, and their mixture.

10. The method of claim 5, wherein the functionalizing of the betulin comprises preparing a first intermediate from the betulin; and functionalizing the first intermediate with hydrophilic moieties.

11. The method of claim 10, further comprising preparing a second intermediate from first intermediate; and functionalizing the second intermediate with hydrophilic moieties, wherein the preparing the second intermediate comprises activating C3-hydroxyl group of the first intermediate to chloroformate.

12. The method of claim 11, wherein C3-hydroxyl group of the betulin is functionalized with a hydrophilic group in the step of functionalizing the second intermediate.

13. The method of claim 10, wherein C28-hydroxyl group of the betulin is functionalized with a hydrophilic group in the step of functionalizing the first intermediate.

14. The method of claim 9, wherein the hydrophilic moieties is selected from polyether glycol (PEG), polyetheramine (PEA), sugar, saccharide, oligo-saccharide, glucosamine, chitosan, and oligo-chitosan.

15. A surfactant composition comprising the compound of claim 1.
